(19) United States
(12) Patent Application Publication
(10) Pub. No.: US 2020/0406054 A1
(43) Pub. Date: Dec. 31, 2020

(54) LIGHT EMISSION CONTROL DEVICE FOR INHIBITING ACCUMULATION OF AMYLOID BETA PLAQUE

(71) Applicant: COLOR SEVEN CO., LTD., Seoul (KR)

(72) Inventors: Nam Gyun KIM, Jeonju-si, Jeollabuk-do (KR); Kyong Jun PARK, Seoul (KR); Yong II SHIN, Busan (KR)

(21) Appl. No.: 16/970,064

(22) PCT Filed: Dec. 13, 2018
(86) PCT No.: PCT/KR2018/015807

§ 371 Date: Aug. 14, 2020

(30) Foreign Application Priority Data
Feb. 21, 2018 (KR) 10-2018-0020773

(51) Int. Cl. A61N 5/06

(52) U.S. Cl. CPC A61N 5/0613; A61N 2005/0662; A61N 2005/0643; A61N 2005/0626

(57) ABSTRACT

The present invention relates to a light emission control device for inhibiting accumulation of amyloid beta plaques, wherein light emission is controlled whereby nerve endings distributed in the epidermis or dermis layer under the skin surface within a specific region of the neck and the nape of the neck in an animal or human body are simulated with visible light energy for a predetermined period of time, and nitrergic nerve terminals connected to the stimulated nerve endings and the nervous system are induced to secrete a material, such as nitric oxide, which in turn relaxes the cerebral blood veins or lymphatic vessels in contact with the nitrergic nerve terminals to induce cerebral blood circulation or lymph circulation enhancement, thereby inhibiting the accumulation of amyloid beta plaques.

### TECHNICAL FIELD

The present invention relates to a light emission control device for inhibiting an accumulation of amyloid beta plaque, and more specifically, to a light emission control device for inhibiting an accumulation of amyloid beta plaque, which controls light irradiation to inhibit an accumulation of amyloid beta plaque by stimulating nerve endings distributed in an epidermis layer or a dermis layer under a skin surface of a specific area in a neck and a nape of the neck using light energy, relaxing blood vessels or lymphatic vessels in a brain, and repeatedly increasing a brain blood flow or lymph circulation.

### BACKGROUND ART

Alzheimer's dementia is a degenerative brain disease that occurs due to damage to brain nerve cells and leads to hypomnesis and cognitive impairment. An exact cause of the Alzheimer's disease has not been found yet, and there is no therapeutic agent for the Alzheimer's dementia. Amyloid beta plaque has been considered to be a major cause of the Alzheimer's dementia. This is because an accumulation of the amyloid beta plaque has been found in brains of Alzheimer's patients. In actuality, for about 20 years, an amyloid beta hypothesis has been the most central theory in dementia research. When a concentration of amyloid beta is increased, since brain nerve cells are destroyed and memory is eventually erased, it has been learned that an amyloid beta protein is used as an important biomarker for a disease diagnosis when dementia is diagnosed. Therefore, a main cause of the dementia is an increase in amyloid beta plaque, and there is an increasing demand to develop a therapeutic agent that inhibits an accumulation of the beta amyloid plaque. However, for a long time, in order to treat or alleviate symptoms such as the Alzheimer's dementia, a great deal of development research has been conducted on new medicines that inhibit an accumulation of beta amyloid plaque, but through clinical test results, it has been learned that the symptoms are not well treated or reduced using medicine. Medicines currently prescribed to dementia patients are brain function improving agents that delay the progression of dementia rather than treating the dementia.

In recent years, it has been learned that symptoms such as cognitive impairment and Alzheimer's dementia are symptoms that occur due to a lack of supply of oxygen and nutrients to brain nerve cells because blood circulation is not smooth in a brain for a long time due to problems with contraction and relaxation of smooth muscles in blood vessels of the brain. In addition, it has also been learned that diseases such as Alzheimer's dementia occur because metabolic wastes of various cells which come from the brain every day are not properly discharged due to a non-smooth blood or lymph circulation such as to cause an accumulation of amyloid beta plaque.

Therefore, since it has been learned that blood circulation in the brain is closely related to treatment or prevention of symptoms such as cognitive impairment and Alzheimer's dementia, there is a desperate need to develop technology for a new device or method for inhibiting an accumulation of amyloid beta plaque by simply and conveniently inducing an increase in brain blood circulation or lymph circulation, increasing supply of oxygen and nutrients to damaged cells that serve as a clearance system of the brain and gradually restoring the function of the damaged clearance system of the brain.

### DISCLOSURE

### Technical Problem

The present invention is directed to providing a light emission control device for inhibiting an accumulation of amyloid beta plaque, which controls light irradiation to inhibit an accumulation of amyloid beta plaque by stimulating nerve endings distributed in an epidermis layer or a dermis layer under a skin surface of a specific area in a neck and a nape of the neck of an animal or human body for a certain period of time using visible light energy, inducing nitrergic nerve terminals connected to the stimulated nerve endings through a nervous system to secrete a material such as nitric oxide, relaxing brain blood vessels and lymphatic vessels in contact with the nitrergic nerve terminals using the secreted material, inducing increases in brain blood circulation and lymph circulation, increasing supply of oxygen and nutrients to damaged cells that serve as a clearance system of a brain, and gradually restoring the function of the damaged clearance system of the brain.

### Technical Solution

According to an embodiment of the present invention, a light emission control device for inhibiting an accumulation of amyloid beta plaque includes a light irradiator in which a light filter and a light source configured to emit visible light in a visible light wavelength band are embedded and which emits the visible light emitted from the light source through one surface of the light irradiator, and an attacher, wherein, in a state in which the light irradiator is directly attached or adjacent to a skin area of a specific area in a neck and a nape of the neck of an animal or human body by the attacher, the light irradiator is controlled to emit the visible light for a set time for light irradiation and controlled to stop the emission of the visible light after the set time has elapsed, and the visible light inhibits an accumulation of amyloid beta plaque by stimulating nerve endings distributed in an epidermis layer or a dermis layer under a skin surface of a corresponding area for a certain period of time, induces nitrergic nerve terminals connected to the stimulated nerve endings through a nervous system to secrete a material such as nitric oxide, relaxes brain blood vessels and lymphatic vessels in contact with the nitrergic nerve terminals using the secreted material, induces increases in brain blood circulation and lymph circulation, increases supply of oxygen and nutrients to damaged cells that serve as a clearance system of a brain, and gradually restores the function of the damaged clearance system of the brain.

In a state in which the light irradiator is directly attached or adjacent to a skin surface positioned in one area of each of left and right vertebral arteries in the nape of the neck of the animal or human body or a skin surface positioned in one area of each of left and right carotid arteries in the neck by the attacher, the accumulation of the amyloid beta plaque may be inhibited by stimulating nerve endings, which are distributed in a dermis layer or an epidermis layer under the skin surface positioned in the vertebral artery or the carotid artery, for a set time using the visible light emitted from the light irradiator, inducing nitrergic nerve terminals around the brain blood vessels connected to the stimulated nerve endings through peripheral nerves to secrete the material such as the nitric oxide through neuronal nitric oxide synthase (nNOS), relaxing the brain blood vessels and the lymphatic vessels in contact with the nitrergic nerve terminals using the secreted material, repeatedly inducing the increases in brain blood circulation and lymph circulation, increasing the supply of the oxygen and the nutrients to the damaged cells that serve as the clearance system of the brain, and gradually restoring the function of the damaged clearance system of the brain.

The nerve endings, which are distributed in the dermis layer or the epidermis layer under the skin surface positioned in the vertebral artery or the carotid artery, may be stimulated for 10 minutes to 30 minutes once a day using the visible light emitted from the light irradiator.

Light irradiation of the light irradiator may be controlled such that the light irradiator emits light having a peak wavelength ranging from 590 nm to 620 nm and an intensity ranging from 1 mW/cm² to 5 mW/cm².

User information such as a light irradiation method, a light irradiation use history, remaining battery capacity, and a remaining light irradiation time may be provided to a user through an embedded display, and a light irradiation start button and a light irradiation end button may be displayed on the embedded display to allow the user to control light irradiation of the light irradiator.

The light irradiator may be allowed to emit light for the set time for light irradiation at a time interval (for example, at an interval of eight or six hours) set by a preset program, and after the set time for light irradiation has elapsed, a light irradiation end signal may be automatically output to allow the light irradiator to stop the emission of the light.

### Advantageous Effects

By utilizing the present invention, visible light can be repeatedly irradiated onto a surface area of a specific region in a neck and a nape of the neck of an animal or human body for a set time every day for several months (for example, three months to six months) to repeatedly induce an increase in blood circulation or lymph circulation in a brain through relaxation of blood vessels and lymphatic vessels and increase supply of oxygen and nutrients to damaged cells that serve as a clearance system of the brain through the increase in blood circulation or lymph circulation in the brain, and gradually restore the function of the damaged clearance system of the brain, thereby discharging amyloid beta plaque to prevent or treat Alzheimer's disease.

### DESCRIPTION OF DRAWINGS

FIG. 1 illustrates a state of use of a light emission control device for inhibiting an accumulation of amyloid beta plaque according to an embodiment of the present invention.

FIG. 2 illustrates a state of use of the light emission control device for inhibiting an accumulation of amyloid beta plaque according to another embodiment of the present invention.

FIG. 3 illustrates states in which a light irradiator controlled by the light emission control device for inhibiting an accumulation of amyloid beta plaque is attached to a nape of an animal's neck and a nape of a human's neck according to an embodiment of the present invention.

FIG. 4 is a diagram for describing a protocol of an experiment on controlling light irradiation to inhibit an accumulation of amyloid beta plaque according to the present invention.

FIG. 5 shows graphs of results of a Morris water maze strength-of-memory test in an experiment on controlling light irradiation to inhibit an accumulation of amyloid beta plaque on an animal model according to the present invention.

FIG. 6 shows graphs of results of an elevated plus maze test in an experiment on controlling light irradiation to inhibit an accumulation of amyloid beta plaque on an animal model according to the present invention.

FIG. 7 shows graphs of results of a thioflavin (Aβ plaque) staining test in an experiment on controlling light irradiation to inhibit an accumulation of amyloid beta plaque on an animal model according to the present invention.

### MODES OF THE INVENTION

Hereinafter, exemplary embodiments of the present invention will be described in more detail with reference to the accompanying drawings.

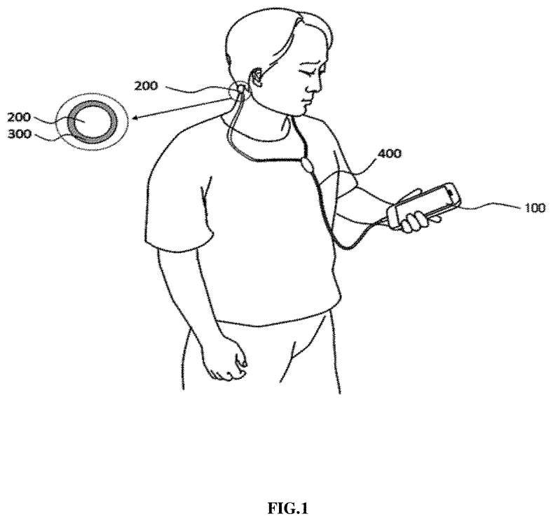

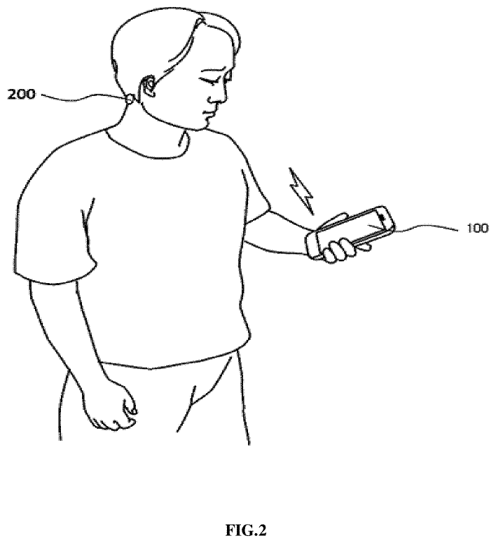

Referring to FIGS. 1 to 2, a light emission control device 100 for inhibiting an accumulation of amyloid beta plaque controls light irradiation of a light irradiator 200.

The light emission control device 100 may control light irradiation of the light irradiator 200, which is directly attached or adjacent to a skin area of a specific area in a neck and a nape of the neck of an animal or human body by an attacher 300 formed in the form of double-sided tape, an adsorption tool, head gear, or a necklace mounted on an ear or head, through a wire 400 as shown in FIG. 1 or in an wireless manner as shown in FIG. 2.

For reference, FIG. 1 illustrates a state in which the light irradiator 200 is directly attached to a skin area of a human by the attacher 300 formed of the double-sided tape.

In order to control light irradiation of the light irradiator 200, the light emission control device 100 includes a display, a light irradiation control microprocessor or light irradiation control circuit, a wireless transceiver, a power supply device, a control device on-off switch, a light irradiation operation indicating lamp (for example, a light-emitting diode (LED)), an alarm device, and the like.

The light emission control device 100 may be manufactured as a portable type (for example, a smart communication device such as an Android phone, an iPhone, or a smartwatch) so that a user may carry the light emission control device 100 to control light irradiation of the light irradiator 200 by himself or herself.

The light emission control device 100 may be robustly manufactured in a large size to be installed in a hospital and thus may also be used for the hospital so that an animal or patient visits the hospital to receive light irradiation using the light irradiator 200.

When the attacher 300 is formed in the form of the head gear mounted on an ear or head, the light emission control device 100 may be embedded and installed in a portion of a body of the head gear. In this case, the light emission control device 100 may be connected to a smart communication device such as an Android phone, an iPhone, or a smartwatch in a wired manner or a wireless manner through a wireless communication method and thus may be controlled using the smart communication device to control light irradiation of the light irradiator 200.

When the attacher 300 is formed in the form of the necklace, the light emission control device 100 may be embedded and installed in a portion of a body of the necklace. In this case, the light emission control device 100 may be connected to a smart communication device such as an Android phone, an iPhone, or a smartwatch in a wired manner or a wireless manner through a wireless communication method and thus may be controlled using the smart communication device to control light irradiation of the light irradiator 200.

In a state in which the light irradiator 200, which is embedded with a light source configured to emit visible light in a visible light wavelength band and a light filter and emits visible light emitted from the light source through one surface thereof, is directly attached or adjacent to a skin area of a specific area in a neck and a nape of the neck of an animal or human body by the attacher 300, the light emission control device 100 controls the light irradiator 200 to emit visible light so as to inhibit an accumulation of amyloid beta plaque by stimulating nerve endings distributed in an epidermis layer or a dermis layer under the skin surface of the specific area for a certain period of time, inducing nitrergic nerve terminals connected to the stimulated nerve endings through a nervous system to secrete a material such as nitric oxide, relaxing brain blood vessels and lymphatic vessels in contact with the nitrergic nerve terminals using the secreted material, inducing increases in brain blood circulation and lymph circulation, increasing supply of oxygen and nutrients to damaged cells that serve as a clearance system of a brain, and gradually restoring the function of the damaged clearance system of the brain. In addition, the light emission control device 100 controls the light irradiator 200 to emit the visible light for a set time for light irradiation and end the emission of the visible light after the set time has elapsed.

In a state in which the light irradiator 200 is directly attached or adjacent to a skin surface positioned in one area of each of left and right vertebral arteries in a nape of a neck of an animal or human body or a skin surface positioned in one area of each of left and right carotid arteries in the neck by the attacher 300, the light emission control device 100 inhibits an accumulation of amyloid beta plaque by stimulating nerve endings, which are distributed in a dermis layer or an epidermis layer under the skin surface positioned in the vertebral artery or the carotid artery, for a set time using visible light emitted from the light irradiator 200, inducing nitrergic nerve terminals around brain blood vessels connected to the stimulated nerve endings through peripheral nerves to secrete a material such as nitric oxide through neuronal nitric oxide synthase (nNOS), relaxing the brain blood vessels and lymphatic vessels in contact with the nitrergic nerve terminals using the secreted material, repeatedly inducing increases in brain blood circulation and lymph circulation, increasing supply of oxygen and nutrients to damaged cells that serve as a clearance system of a brain, and gradually restoring the function of the damaged clearance system of the brain.

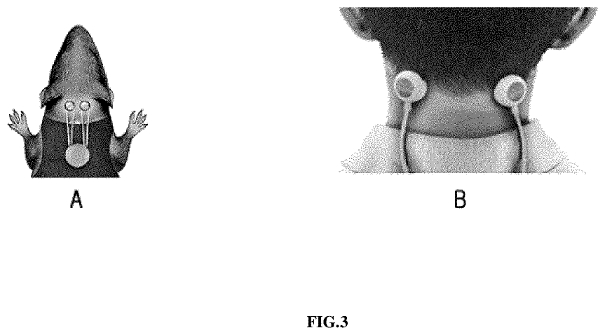

For reference, FIG. 3 illustrates states in which the light irradiator 200 controlled by the light emission control device 100 is attached to positions of left and right vertebral arteries in a nape of an animal's neck (see A of FIG. 3) and is attached to positions of left and right vertebral arteries in a nape of a human's neck (see B of FIG. 3) according to an embodiment of the present invention.

Unlike that shown in FIG. 3, the light irradiator 200 may be used in a state of being attached or adjacent to left and right carotid arteries in a neck of an animal or human body.

In order to inhibit an accumulation of amyloid beta plaque by relaxing brain blood vessels or lymphatic vessels, the light emission control device 100 stimulates nerve endings, which are distributed in a dermis layer or an epidermis layer under a skin surface positioned in a vertebral artery or a carotid artery, for 10 minutes to 30 minutes once a day using visible light emitted from the light irradiator 200.

For reference, when light is repeatedly irradiated for 10 minutes to 30 minutes once a day for about one month to about six months using the light irradiator 200 according to a symptom of an animal or human utilizing the light emission control device 100, brain blood vessels or lymphatic vessels can be relaxed so as to inhibit an accumulation of amyloid beta plaque.

The light emission control device 100 controls light irradiation of the light irradiator 200 such that the light irradiator 200 emits light having a peak wavelength ranging from 590 nm to 620 nm and an intensity ranging from 1 mW/cm² to 5 mW/cm².

In the light irradiator 200 of which light irradiation is controlled by the light emission control device 100, light, which is emitted from the light source formed in the form of one of an LED, an organic-light emitting diode (OLED), and a micro LED, or a combination thereof, may have a peak wavelength ranging from 590 nm to 620 nm, and light emitted through a light-emitting opening formed in one surface of the light irradiator 200 may have an intensity ranging from 1 mW/cm² to 5 mW/cm².

The light emission control device 100 provides user information such as a light irradiation method, a light irradiation use history, remaining battery capacity, and a remaining light irradiation time to a user through an embedded display and displays a light irradiation start button and a light irradiation end button on the embedded display to allow the user to control light irradiation of the light irradiator 200.

When the light irradiation start button is selected, the light emission control device 100 outputs a light irradiation driving signal to control the light irradiator 200 to emit light for a set time for light irradiation. After the set time for light irradiation has elapsed, the light emission control device 100 automatically outputs a light irradiation end signal to control the light irradiator 200 to stop the emission of light.

When the light irradiation end button is selected while the light irradiator 200 is irradiating light, the light emission control device 100 controls the light irradiator 200 to stop the emission of light.

The light emission control device 100 allows the light irradiator 200 to emit light for a set time for light irradiation at a time interval (for example, at an interval of eight or six hours) set by a preset program. After the set time for light irradiation has elapsed, the light emission control device 100 automatically outputs a light irradiation end signal to allow the light irradiator 200 to stop the emission of light.

By using the light emission control device 100 for inhibiting an accumulation of amyloid beta plaque according to the present invention configured as described above, as a result of an experiment on controlling light irradiation of the light irradiator 200 on a vertebral artery of a Alzheimer's animal model, it was confirmed that a spatial memory ability of the Alzheimer's animal model was improved and an accumulation of amyloid beta plaque (Aβ plaque) was inhibited.

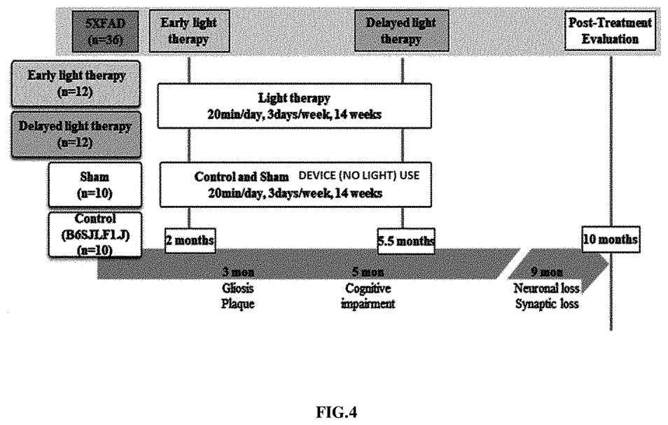

FIG. 4 is a diagram for describing a protocol of an experiment on controlling light irradiation to inhibit an accumulation of Aβ plaque according to the present invention.

As shown in FIG. 4, as an Alzheimer's animal model, a 5XFAD animal model, in which Aβ plaque began to be generated at three months and cognitive impairment began to appear at five months, was used. An experiment was performed on four groups, such as an early light therapy group (n=12), a delayed light therapy group (n=12), a sham group (n=10), and a control group (n=10). The control group included B6SJL mice of the same age.

Light was irradiated onto the early light therapy group for 20 minutes a day, three times a week, and for 14 weeks from two months before Aβ plaque is generated, and light was irradiated onto the delayed light therapy group for 20 minutes a day, three times a week, and for 14 weeks from 5.5 months when Aβ plaque was generated and cognitive impairment appeared. In addition, a Morris water maze test for evaluation of a cognitive ability and a spatial memory and for verification evaluation of Aβ plaque inhibition, an elevated plus maze test, and thioflavin (Aβ plaque)-S staining were performed at 10 months.

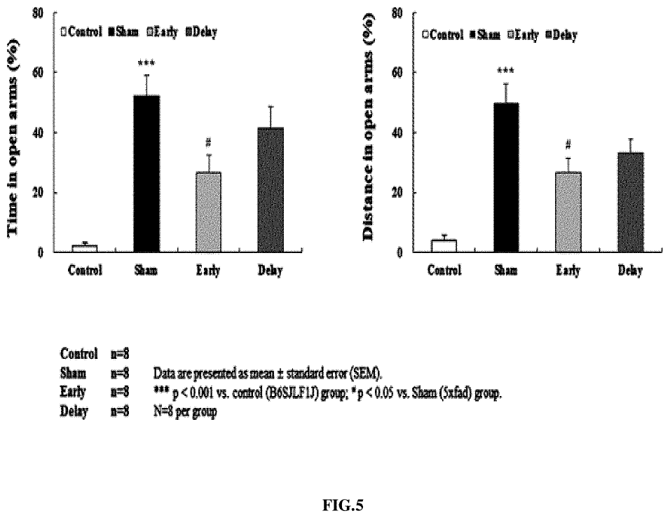

FIG. 5 shows graphs of results of a Morris water maze strength-of-memory test in an experiment on controlling light irradiation to inhibit an accumulation of Aβ plaque on an animal model according to the present invention.

As shown in FIG. 5, it could be confirmed that, as compared with the sham group, a long-term learning ability and a spatial memory ability were improved in both the early light therapy group and the delayed light therapy group.

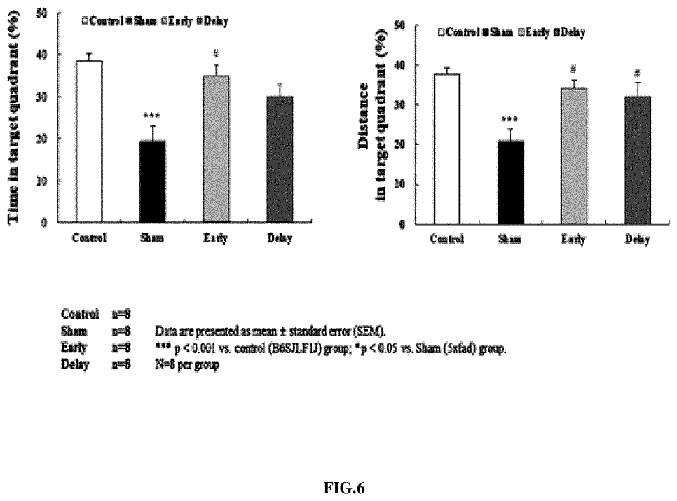

FIG. 6 shows graphs of results of an elevated plus maze test in an experiment on controlling light irradiation to inhibit an accumulation of Aβ plaque on an animal model according to the present invention.

As shown in FIG. 6, it could be confirmed that, as compared with the sham group, behavior related to anxiety was improved in both the early light therapy group and the delayed light therapy group.

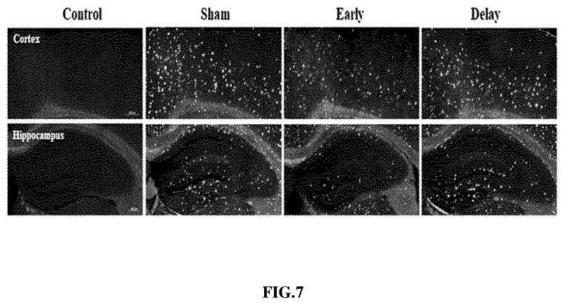

FIG. 7 shows graphs of results of a thioflavin (Aβ plaque) staining test in an experiment on controlling light irradiation to inhibit an accumulation of Aβ plaque on an animal model according to the present invention.

As shown in FIG. 7, it could be confirmed that, as compared with the sham group, Aβ plaque was not considerably accumulated in both areas of a cerebral cortex and a hippocampus of the early light therapy group, and an accumulation of Aβ plaque was slightly improved in the delayed light therapy group.

As described above, by utilizing the present invention, visible light can be repeatedly irradiated onto a surface area of a specific region in a neck and a nape of the neck of an animal or human body for a set time every day for several months (for example, three months to six months) to repeatedly induce an increase in blood circulation or lymph circulation in a brain through relaxation of blood vessels and lymphatic vessels and increase supply of oxygen and nutrients to damaged cells that serve a clearance system of the brain through the increase in blood circulation or lymph circulation in the brain and gradually restore the function of the damaged clearance system of the brain, thereby discharging Aβ plaque to prevent or treat Alzheimer's disease.

A light emission control device for inhibiting an accumulation of Aβ plaque described above is not limited to the above embodiments, and various modifications may be made therein by those of ordinary skill in the art without departing from the technical scope claimed in the following claims.

1. A light emission control device for inhibiting an accumulation of amyloid beta plaque, the light emission control device comprising:
a light irradiator in which a light filter and a light source configured to emit visible light in a visible light wavelength band are embedded and which emits the visible light emitted from the light source through one surface of the light irradiator; and
an attacher,
wherein, in a state in which the light irradiator is directly attached or adjacent to a skin area of a specific area in a neck and a nape of the neck of an animal or human body by the attacher, the light irradiator is controlled to emit the visible light for a set time for light irradiation and controlled to stop the emission of the visible light after the set time has elapsed, and
the visible light inhibits an accumulation of amyloid beta plaque by stimulating nerve endings distributed in an epidermis layer or a dermis layer under a skin surface of a corresponding area for a certain period of time, induces nitrergic nerve terminals connected to the stimulated nerve endings through a nervous system to secrete a material such as nitric oxide, relaxes brain blood vessels and lymphatic vessels in contact with the nitrergic nerve terminals using the secreted material, induces increases in brain blood circulation and lymph circulation, increases supply of oxygen and nutrients to damaged cells that serve as a clearance system of a brain, and gradually restores the function of the damaged clearance system of the brain.

2. The light emission control device of claim 1, wherein, in a state in which the light irradiator is directly attached or adjacent to a skin surface positioned in one area of each of left and right vertebral arteries in the nape of the neck of the animal or human body or a skin surface positioned in one area of each of left and right carotid arteries in the neck by the attacher, the accumulation of the amyloid beta plaque is inhibited by stimulating nerve endings, which are distributed in a dermis layer or an epidermis layer under the skin surface positioned in the vertebral artery or the carotid artery, for a set time using the visible light emitted from the light irradiator, inducing nitrergic nerve terminals around the brain blood vessels connected to the stimulated nerve endings through peripheral nerves to secrete the material through neuronal nitric oxide synthase (nNOS), relaxing the brain blood vessels and the lymphatic vessels in contact with the nitrergic nerve terminals using the secreted material, repeatedly inducing the increases in brain blood circulation and lymph circulation, increasing the supply of the oxygen and the nutrients to the damaged cells that serve as the clearance system of the brain, and gradually restoring the function of the damaged clearance system of the brain.

3. The light emission control device of claim 2, wherein the nerve endings, which are distributed in the dermis layer or the epidermis layer under the skin surface positioned in the vertebral artery or the carotid artery, are stimulated for 10 minutes to 30 minutes once a day using the visible light emitted from the light irradiator.

4. The light emission control device of claim 1, wherein light irradiation of the light irradiator is controlled such that the light irradiator emits light having a peak wavelength ranging from 590 nm to 620 nm and an intensity ranging from 1 mW/cm2 to 5 mW/cm2.

5. The light emission control device of claim 1, wherein user information is provided to a user through an embedded display, and
a light irradiation start button and a light irradiation end button are displayed on the embedded display to allow the user to control light irradiation of the light irradiator.

6. The light emission control device of claim 1, wherein the light irradiator is allowed to emit light for the set time for light irradiation at a time interval set by a preset program, and
after the set time for light irradiation has elapsed, a light irradiation end signal is automatically output to allow the light irradiator to stop the emission of the light.

7. The light emission control device of claim 1, wherein the user information is selected from a light irradiation method, a light irradiation use history, remaining battery capacity, and a remaining light irradiation time.
